(19) United States
(12) Patent Application Publication
(10) Pub. No.: US 2002/0114850 A1
(43) Pub. Date: Aug. 22, 2002

(54) Therapeutic bath solution containing alkali metal hypohalite

(76) Inventors: Jurdon Wayne Camper, Rolla, ND (US); Marilyn A. Delorme, Bellcourt, ND (US); John E. Festvog, Rolla, ND (US)

Correspondence Address: DORSEY & WHITNEY LLP, 801 GRAND, SUITE 3900, DES MOINES, IA, 50309, US

(21) Appl. No.: 09/784,215

(22) Filed: Feb. 16, 2001

(51) Int. Cl. A61K 33/14

(52) U.S. Cl. 424/665; 424/661

(57) ABSTRACT

A therapeutic bath solution containing a mixture of water and an alkali metal hypohalite (e.g., sodium hypochlorite). Optionally, the solution also contains an odor masking agent. The user soaks his or her body in the bath solution for up to 30 minutes. The alkali metal hypohalite penetrates into the subcutaneous tissue of the user through the skin pores and lymph nodes. Once in the body, the alkali metal hypohalite stimulates the immune system and alleviates aches, pains, inflammation, and other symptoms commonly caused by microbial (e.g., viral, bacterial, fungal, or parasitic) infection, arthritis, allergies, hemorrhoids, and a host of other ailments.

### FIELD OF THE INVENTION

The instant invention relates to a therapeutic bath. More specifically, the invention relates to a therapeutic bath solution that contains an alkali metal hypohalite (AMH) and delivers it to the subcutaneous tissue and, thereby, stimulates the immune system to treat, and in some cases cure, a variety of ailments including: viral infections (e.g., colds, HIV infection); microbial infections, dermatitis; Lyme disease, allergies; hemorrhoids; arthritis; and gout.

### BACKGROUND ART

The most common AMH is sodium hypochlorite (NaOCl). Sodium hypochlorite is the active agent in household bleach. Due to its bactericidal properties, it is also a common ingredient in swimming pool disinfectants and water purification treatments. See Hawley's Condensed Chemical Dictionary 13th ed., by Richard R. Lewis, John Wiley & Sons, New York, N.Y., page 1019 (1997). However, sodium hypochlorite is used in extremely low concentration for these purposes.

A number of compositions containing sodium hypochlorite have been patented. Representative patents include U.S. Pat. Nos.: 4,737,307; 4,927,641; 5,273,678; 5,472,714; and 5,427,801.

U.S. Pat. No. 4,737,307 is directed to a skin cleanser capable of removing smegma and surface bacteria fungus and viruses from the skin. The cleanser is an aqueous solution of cetylpyridinium chloride, chlorine dioxide, sodium hypochlorite, polyoxyethylene (20) sorbitan monostearate and sodium benzoate.

U.S. Pat. No. 4,927,641 is directed to a veterinary liniment. The liniment comprises dimethlysulfoxide and sodium hypochlorite. The liniment is preferably a thin liquid but may be in the form of a lotion, gel, or cream.

U.S. Pat. No. 5,273,678 is directed to a stable aqueous solution containing sodium hypochlorite, a pH regulator, and water. The solution is used as an antiseptic.

U.S. Pat. Nos. 5,472,715 and 5,427,801 are directed to an antifungal agent for the treatment of surface skin diseases such as athlete's foot, ringworm and tinea caused by dermatophytes, eczema, tinea or various fungi. The agent comprises a detergent solution of sodium hypochlorite, sodium sulfite, sodium nitrate, sodium chlorate, potassium chlorate, hydrogen peroxide, oxone water, sodium nitrite, potassium nitrite, nonionic surface active agent, and water.

None of these references teach a high concentration therapeutic bath solution that provides an in vivo treatment of disease with AMH. Furthermore, with the exception of U.S. Pat. No. 4,927,641, all of these references are directed to compositions wherein the active ingredient remains in the epidermal layer of the skin, i.e., the uppermost layer of skin cells, as opposed to penetrating into the subcutaneous tissue. None of these references recognize the ability of an AMH be absorbed by the body and, once therein, to stimulate the immune system and alleviate the aches, pains, inflammation, itching, and other symptoms of common maladies such as microbial infection, allergies, hemorrhoids, and arthritis.

There are a number of antimicrobial (e.g., antiviral, antibacterial, antifungal, antiparasitic) treatments on the market for treating infections in subcutaneous tissue. In addition, there is a large body of art dedicated to treating internal ailments such as hemorrhoids, arthritis and allergies. However, most of the treatments that target these maladies are delivered either orally (i.e., in a pill, capsule or liquid) or intravenously (i.e., by injection). These delivery methods, while effective, are problematic. Intravenous delivery can be difficult and is relatively costly. Oral delivery is sometimes hampered by the foul taste of the active agents employed.

In addition, many of these treatments cause side effects. Common side effects include fatigue, raised blood pressure, skin irritation, and a weakened immune system.

### SUMMARY OF THE INVENTION

The present invention comprises a bath solution in which the user soaks for a predetermined amount of time. The user soaks in the bath for 30 minutes every other day.

The bath contains a relatively high concentration of AMH, e.g., sodium hypochlorite, which enters the user's body (i.e., into the subcutaneous tissue) through the pores and lymph nodes. Once in the user's system, the AMH stimulates the immune system and treats, and in some cases cures, a variety of ailments including microbial (viral, bacterial, fungal and parasitic) infection, and the aches pains and swelling associated with common diseases such as arthritis, dermatitis, etc.

This bath solution does not attack the healthy cells of the user and does not destroy the user's immune system. Instead, this bath solution vitalizes the immune system and, in cooperation with the immune system, effectively alleviates the symptoms of disease.

### DETAILED DESCRIPTION OF THE INVENTION

The bath solution of the instant invention comprises: water; AMH; and, optionally, an odor emitting substance. Preferably, these are the only components of the bath solution.

The preferred AMH is sodium hypochlorite. However, other AMHs, such as calcium hypochlorite, are suitable.

To make the bath solution, approximately 0.33 ounces to 3 ounces (10 to 90 milliliters) of 7.25%, by volume, aqueous AMH, is added to every gallon (3.77 liters) of water. This roughly corresponds to a bath solution containing about 0.02% to about 0.17% of pure AMH in the bath solution. Note that the concentration of pure AMH in the bath solution is almost 1000 fold higher than the concentration of AMH in community pools, etc. . . . In fact, baths with a concentration of pure AMH as high as about 6.33% by volume are both safe and effective.

Preferably, thirty-two gallons of water (121 liters) and 1 quart (circa 1.1 liters) of 7.25%, by volume, aqueous solution of sodium hypochlorite are employed. This roughly corresponds to a bath solution containing about 0.065% of pure AMH and, generally, provides enough bath solution to adequately submerge the user.

The amount of bath solution is not critical. However, for maximum benefit, it is preferred to immerse the patient in solution leaving only the face exposed. Generally, at least 30 gallons of bath solution are necessary.

The odor emitting substance serves to neutralize, or mask, the unpleasant smell of the AMH. Any substance that provides a pleasant odor, e.g. floral or fruit smell, can be employed.

Preferably, the AMH is the principal active therapeutic agent in the bath solution. More preferably, the AMH is the only active therapeutic agent in the bath solution.

In practice, the user submerges his or her body in the bath solution for at least 30 minutes every other day. Thirty minutes is sufficient time for the AMH to penetrate into the body and take effect. More frequent applications are not generally recommended given the strength of the bath. Obviously, the number and frequency of baths that are necessary depends upon the severity and type of ailment.

As the user soaks in the solution, the AMH enters the user's body through the skin's pores and lymph nodes. Once in the subcutaneous tissue, the AMH treats the symptoms commonly caused by microbial infections, allergies, arthritis, hemorrhoids, and other ailments.

The solution does not adversely affect the user's immune system or healthy cells. Instead, sodium hypochlorite actually vitalizes the user's immune system and helps alleviate the symptoms commonly exhibited by microbial infections, arthritis, hemorrhoids, allergies, etc. . . . In other words, the sodium hypochlorite alleviates aches, pains, inflammation, and other common symptoms of disease.

Treatment with the therapeutic bath solution may be done in conjunction with other treatments. This is especially true when the other treatment is the daily application of an AMH containing lotion. Such a lotion is described in detail in a concurrently filed and copending application to the same inventors.

The following examples illustrate a preferred embodiment of the invention and the efficacy of the same in treating a variety of ailments:

### EXAMPLE 1

Aches, Pains, and Swelling

A 59 year old woman was diagnosed as having severe aches and pain. She was serum positive, and had severe deformities of her hands and chronic pain in her shoulders, knees, left elbow, lower back and feet. She was classified as 80% disabled. She soaked in a bath solution comprising water, sodium hypochlorite, and a floral odor-emitting substance for approximately 30 minutes. She did this every other day for a total of ten times. At the end of the treatment, the swelling in her hands receded. Furthermore, the chronic pain affecting her joints disappeared.

### EXAMPLE 2

Dermatitis

A 50 year old man was diagnosed with acute dermatitis. It affected his right foot and had spread over 80 percent of his exposed skin. After receiving just three bath solution treatments, containing the components and concentrations described in example 1, the dermatitis receded. After two weeks, there were no visible signs of a dermatitis problem.

### EXAMPLE 3

Viral Infection

A 57 year old man was diagnosed as having a viral infection. His Prostrate Specific Antigen (PSA) level was measured at 20.4. Note that normal PSA levels are less than 4.0 ng/ml. After several treatments with the bath solution, containing the components and concentrations detailed in example 1, his PSA level was 2.3.

### EXAMPLE 4

Bells Palsy

A 59 year old male suffering from Bells Palsy was treated in a 30 minute bath every other day for 16 days. At the end of the treatment the Bells Palsy was no longer active.

### EXAMPLE 5

Multiple Sclerosis

A 55 year old female with a history of Multiple Sclerosis suffered from lost muscle tone, numbness, and pain. She was not able to walk without assistance. For 28 days she soaked 30 minutes every other day in a bath solution of sodium hypochlorite and water (using 1 quart of 7.25%, by volume, sodium hypochlorite per 32 gallons of water.) In addition, the patient spread affected areas with a lotion containing sodium hypochlorite. The lotion consists of 60 ml of 7.25%, aqueous sodium hypochlorite solution and 532 ml of ST. IVE'S THERAPY SOLUTION™.¹ After the treatment, she reported diminished pain and numbness and was able to walk unassisted. ¹This lotion is one embodiment of a concurrently filed and copending patent application to the same inventors.

### EXAMPLE 6

HIV

A 50 year old female was diagnosed positive for HIV virus nine years prior to treatment. She had a CD4 level of around 130. The patient soaked every other day for a period of two weeks in the bath solution of example 5 and applied the lotion of example 5 daily to her lymph nodes and around the olfactory nerves. Within a few months her CD4 count was over 600. Note that a normal CD4 count is between 401 and 1532.

### EXAMPLE 7

Lyme Disease

A 67 year old female was treated for Lyme disease. The patient soaked every other day, for a total of four times, in the bath solution of sodium hypochlorite and water described in example 5. In addition, the patient applied the lotion described in example 5 every day for one month. The symptoms of Lyme disease are no longer apparent.

### EXAMPLE 8

Amyotrophic Lateral Sclerosis (ALS—Lou Gehrig's Disease)

A 54 year old white male diagnosed with ALS was suffering from continual cramping. The patient was treated with 30 minute soaks, every other day for four days, in the bath solution of sodium hypochlorite and water described in example 5. Additionally, the lotion described in example 5 was applied every day for a week. After treatment, the cramping stopped completely.

While the invention has been described with reference to exemplary embodiments thereof, those skilled in the art will be able to make various modifications to the described embodiments without departing from the true spirit and scope of the invention as defined in the following claims and their equivalents.

What is claimed is:

1. A therapeutic bath comprising water and alkali hypohalite, wherein the alkali hypohalite is the principal therapeutically active component, and wherein the concentration of the alkali hypohalite is between about 0.02% to about 6.33%, by volume, of the entire bath.

2. The composition of claim 1, wherein the alkali hypohalite is sodium hypochlorite.

3. The composition of claim 1, additionally comprising an odor neutralizing, or masking, agent.

4. The composition of claim 1, containing at least 30 gallons of water.

5. The composition of claim 1, wherein the alkali hypohalite is the only therapeutically active component.

6. A method of treating the aches, pains, and inflammation associated with microbial infection, allergies, hemorrhoids, arthritis, and other maladies, comprising the following steps:
(i) providing a mixture of alkali hypohalite and water, wherein the alkali hypohalite is present in a concentration between about 0.02% to about 6.33%, by volume, of the mixture;
(ii) at least partially immersing a person in said mixture for a time sufficient to permit the alkali hypohalite to penetrate into the subcutaneous tissue through skin pores and lymph nodes.

7. The method of claim 6, wherein the alkali hypohalite is sodium hypochlorite.

8. The method of claim 6, wherein the mixture additionally comprises an odor neutralizing, or masking, agent.

9. The method of claim 6, wherein sufficient mixture is employed to submerge all but the head of the person.

10. The method of claim 6, wherein the mixture contains at least 30 gallons of water.

11. The method of claim 6, wherein the alkali hypohalite is the principal therapeutically active component.

12. The method of claim 6, wherein the person soaks in said mixture for at least 30 minutes.
